(19) United States
(12) Patent Application Publication
(10) Pub. No.: US 2010/0125216 A1
(43) Pub. Date: May 20, 2010

(54) ACTIVITY MONITORING SYSTEM AND MONITORING DEVICE OF THE SAME

(75) Inventors: Bo-Jau Kuo, Taipei (TW); Guo-She Lee, Taipei (TW); Ching-Hsiu Yang, Taipei (TW)

Correspondence Address: HOGAN & HARTSON L.L.P., 1999 AVENUE OF THE STARS, SUITE 1400, LOS ANGELES, CA, 90067, US

(73) Assignee: National Yang-Ming University, Taipei (TW)

(21) Appl. No.: 12/410,481

(22) Filed: Mar. 25, 2009

(30) Foreign Application Priority Data
Nov. 17, 2008 (TW) 097144333

(51) Int. Cl. A61B 5/0402; G01S 1/00

(52) U.S. Cl. 600/509; 342/357.07

(57) ABSTRACT

This invention relates an activity monitoring system and a device thereof. The device includes a global positioning system for providing a positioning information, a wireless communication module, a status sensor module including a multi-axle accelerating sensor and a heart rate variability sensor for sensing subject's activity status and bio-status so as to provide at least one activity information related to the subject and a microprocessor which is coupled to the global positioning system, the wireless communication module and the status sensor module, wherein the microprocessor controls the wireless communication module to send the positioning information and the at least one activity information to the remote host.

### BACKGROUND OF THE INVENTION

1. Field of the Invention

The present invention relates generally to an activity monitoring system and, more particularly, to an activity monitoring system and a device thereof for monitoring bio-information of at least one subject.

2. Prior Art

As the progress of modern medicine, human being's life expectancy is becoming longer each day. On top of that, due to the drastic decrease of number of new born babies in developed countries, caretaking the elders becomes increasingly important to the society. To senior citizens, aging is not only a sign to show how many years they have lived, it also a sign to show their physical performances are gradually deteriorated. Somehow along the way, they begin to feel aching and decrease of movability. To those who do not have relatives or friends to take of, devices or related systems are developed to care for these senior citizens. Based on these systems or devices, specially trained personnel are able to take care of them from a distance. However, all these newly developed systems or devices require additional equipment to match with the new functions. And all these additional equipment are costly and will be a great burden to their financial status.

### SUMMARY OF THE INVENTION

The primary objective of the invention is to provide an activity monitoring system to monitor a subject's bio-information and send the information to a remote host so as to improve the drawbacks of the existing art.

According to one aspect of the invention, an activity monitoring device provided by the invention is to provide including a global positioning system (GPS) for providing a specific position of the subject, a wireless communication module for sending specific information to the microprocessor, a status sensor module including a multi-axle accelerating sensor and a heart rate variability (HRV) sensor for sensing the subject's activity status and bio-status so as to provide all activity sensed information of the subject's and a microprocessor which is coupled to the GPS, the wireless communication module and the status sensor module to control the wireless communication module to send at least one of the positioning information and the sensed activity information to the remote host.

According to another aspect of the invention, an activity monitoring system provided by the invention is to provide an activity monitoring system for monitoring multiple subjects and sending all the information to a remote host. The activity monitoring system includes the remote host and multiple activity monitoring devices to be attached to multiple subjects. Each activity monitoring device includes a global positioning system (GPS) for providing a specific position of the subject, a wireless communication module for sending specific information to the microprocessor, a status sensor module including a multi-axle accelerating sensor and a heart rate variability (HRV) sensor for sensing the subject's activity status and bio-status so as to provide all activity sensed information of the subject's and a microprocessor which is coupled to the GPS, wireless communication module and the status sensor module to control the wireless communication module to send at least one of the positioning information and the sensed activity information to the remote host.

The benefit of the present invention is to provide a low-cost activity monitoring system and a activity monitoring device to provide information concerning the subject's activity, location including longitude and latitude in real-time and analyze them. From the activity information, it is to know the subject's daily variation cycle, sleeping cycle, oxygen consumption rate as well as any micro activity of the subject's body. From the latitude and longitude information, it is able to know the subject's overall activity performance. When multiple subjects are simultaneously monitored by the system, the remote host is able to consolidate the group information to perform an integral analysis and to track all the subjects' activity and locations all at once. When necessary, the system is able to feedback the subjects via an ear set to remind each of the subjects to perform appropriately. Also, the feedback function may also be relayed to caregivers so that the caregivers at a distance may respond to the feedback information promptly. As a result, the system as well as the device of the present invention is able to monitor solitary senior citizens and provide precautions

Other objectives and advantages of the present invention will become obvious to the reader and it is intended that these objectives and advantages are within the scope of the present invention. To the accomplishment of the above and related objectives, this invention may be embodied in the form illustrated in the accompanying drawings, attention being called to the face, however, that the drawings are illustrated only, and that the changes may be made in the specific construction illustrated and described within the scope of this application.

### BRIEF DESCRIPTION OF THE DRAWINGS

FIG. 1 is a schematic view showing the activity monitoring system of the present invention.

FIG. 2 is a block diagram showing the activity monitoring device of the present invention.

FIG. 3 is a schematic view showing that the system of the present invention is employed for tracking multiple subjects' activity.

### DETAILED DESCRIPTION OF THE PREFERRED EMBODIMENT

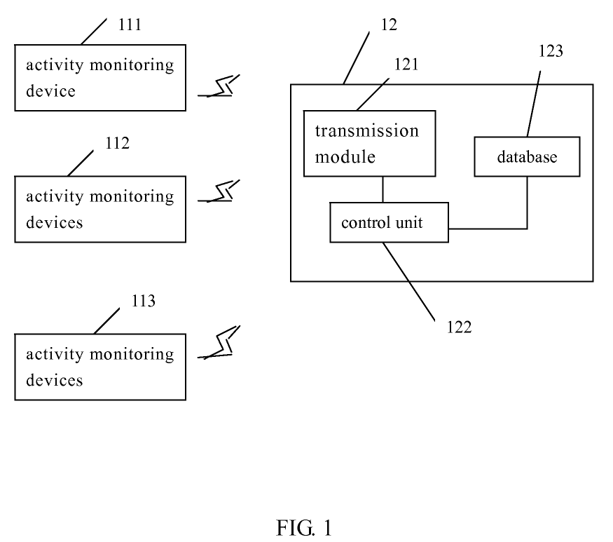

The preferred embodiment of the invention mainly is based on the showing of FIG. 1, wherein the activity monitoring system of the present invention includes multiple activity monitoring devices 111, 112 and 113 and a remote host 12 including a transmission module 121, a control unit 122 and a database 123.

Each of the activity monitoring devices 111, 112 and 113 is mounted on a respective one of subjects, i.e. solitary senior citizens or special group of people, to be monitored. In order to be integrated into our daily lives, it is to be noted that the activity monitoring device of the preferred embodiment is able to be modulated into other devices such as watches, necklaces or any kind of ornamental accessories so that after the activity monitoring device of the present invention is modulated or integrated into such an accessory, others will not even notice the difference.

The remote host 12 is provided in the monitoring center and connected to each of the activity monitoring devices 111, 112, 113 via wireless communication. The control unit 122 is coupled to the transmission module 121 and the database 123. The transmission module 121 may be a GPRS communication module or 3.5 G communication module. The above description should not be interpreted as a limiting to the scope of the present invention; it should only serve as an assisting description to further understand the employment of the invention. The control unit 122 may be a CPU, a computer host of a server.

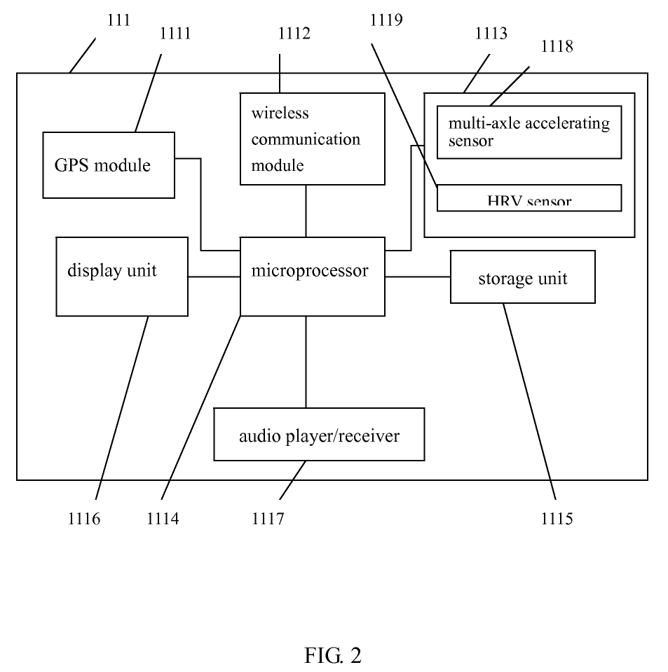

With reference to the block diagram shown in FIG. 2 and still using FIG. 1 for reference, it is to be noted that the activity monitoring device of the present invention includes a global positioning system (GPS) module 1111, a wireless communication module 1112, a status sensor module 1113 including a multi-axle accelerating sensor 1118 and a heart rate variability (HRV) sensor 1119, a microprocessor 1114, a storage unit 1115, a display unit 1116 and an audio player/receiver 1117.

The microprocessor 1114 is coupled to the GPS module 1111 and the wireless communication module 1112, the status sensor module 1113, the storage unit 1115, the display unit 1116 and the audio player/receiver 1117.

In the preferred embodiment of the present invention, it is to be understood that the wireless communication module 1112 may be a GPRS module or a 3.5G module; however, the above example should not be interpreted as a limiting to the scope of the invention and should be serve as assisting explanation to further understanding of the employment of the present invention.

The status sensor module 1113 may be, for example, an accelerometer. The GPS module 1111 is used to provide specific positioning information which includes, for example, latitude and longitude information.

The status sensor module 1113 is used to sense the activity of the subject to be tested. In the examples of the present invention, activity means exercise status and biological status. Further, the multi-axle accelerating sensor 1118 is a 3-axle accelerometer to monitor speed notice signals (accelerating shift and/or RMS notice signal) in three axle, X, Y and Z in real-time, which is, to sense the subject's sudden movement so as to provide activity information related to the tested subject. The HRV sensor 1119 is used to sense the tested subject's biological status, for example, whether the subject is lying still or in agitated situation so as to provide activity information related to the tested subject.

In the preferred embodiment of the present invention, the information sensed and provided by the multi-axle sensor 1118 and HRV sensor 1119 respectively may be independent to one another or may be integrated into one consolidated information. However, the above description is not served as a limiting to the scope of the invention; it only serves as an auxiliary explanation as how the information can be treated in the present invention. The following description will only focus on one activity information for explanation.

In the preferred embodiment of the present invention, the storage unit 1115 is a non-volatile memory for storing positioning information and activity information sensed by the sensor of the preferred embodiment. After the positioning information or an the activity information is stored and reach a predetermined volume or time, the microprocessor 1114 controls the wireless communication module 1112 to send the above information to the remote host 12.

That is, the present invention is not limited by the information volume stored in the storage unit 1115 to determine whether the information should be sent to the remote host 12, it can also be sent based on the predetermined time period, such as daily or weekly. In addition, the storage unit 1115 can be programmed to send out emergency notice signal to designated personnel or institute when there is an emergency or the storage unit 1115 can also be programmed to send out emergency notice signal voluntarily by the subject being evaluated, tested or monitored.

Furthermore, the activity information can be compressed by the microprocessor 1114 and when the compressed information reaches a predetermined volume or a predetermined time period, the microprocessor 1114 controls the wireless communication module 1112 to transmit compressed activity information to the remote host 12.

Other than that, the storage unit 1115 has therein precautionary information and the microprocessor 1114 may constantly compare the activity information with the precautionary information to determine to control the wireless communication module 1112 to send out notice signal to the remote host 12 for help. For example, the precautionary information is programmed to fix the activity information and if the activity information provided by the status sensor module fits the criteria, the subject maybe fainted or lay down on the ground for a while. At the moment, the microprocessor 1114 controls the wireless communication module 1112 to send out a voluntary notice signal to the remote host 12 so that the monitor center is able to respond accordingly.

In other embodiments, the wireless communication module 1112 may test the communication quality at any time. If the subject being evaluated is at a location where the communication quality is good, the wireless communication module 1112 sends out a notice signal to the microprocessor 1114 which, in turn, controls the wireless communication module 1112 to send out at least positioning information and activity information to the remote host 12.

In the embodiments, in order to save electricity during operation of the activity monitoring device 111, the GPS 1111 as well as the wireless communication module 1112 respectively has a first operation status (such as OFF) and a second operation status (such as ON). Normally the GPS 1111 and the wireless communication module 1112 are in the OFF state to save electricity. Under such a circumstance, the transmission of positioning information is initiated or triggered by the activity information.

The microprocessor 1114 will determine whether the activity information is abnormal, such as the subject being evaluated suddenly falls down to the ground for any reason or stays still for a period of time. And when such situation happens, the information picked up by the multi-axle accelerating sensor will remain fixed and no more variation. In the meantime, the microprocessor 1114 will control the wireless communication module 1112 to send out activity information and the positioning information to the remote host 12 in real time so as to save the subject in time.

The remote host 12 can simultaneously receive information (including but not limited to positioning information or activity information) from multiple activity monitoring devices 111, 112, 113 and the remote host 12 further has transmission module 121, control unit 122 and database 123. When the remote host 12 receives at least one of the positioning information and the activity information, the database 123 is used to store to the received information and the remote host 12 analyzes the stored information to determine ability of such as activity and scope of exercise of the subject.

If there is any abnormal situation happens, the remote host 12 controls the transmission module 121 to send a warning message to the activity monitoring device 111 and the warning message may be shown via a display unit 1116 or broadcasted via a audio player/receiver 1117, wherein the warning message may be in the form of a continuously repeated flash or sound.

The remote host 12 of the preferred embodiment primarily functions as an information analysis agent to the subject's daily exercise information and the positioning information and then the information is stored in the storage unit 123 such that when there's an emergency, the remote host 12 is able to send out a warning message in real time.

In addition, the audio player/receiver 1117 includes a microphone and an ear set such that the evaluated subject is able to converse with the remote service personnel via the microphone and ear set of the activity monitoring device 111 and vice versa to form a communication loop for case of emergency.

From the explanation above, it is noted that each of the activity monitoring devices 111, 112, 113 is sending information independently and intermittently so that one remote host 12 is able to monitor up to thousand and even ten thousand activity monitoring devices all at once.

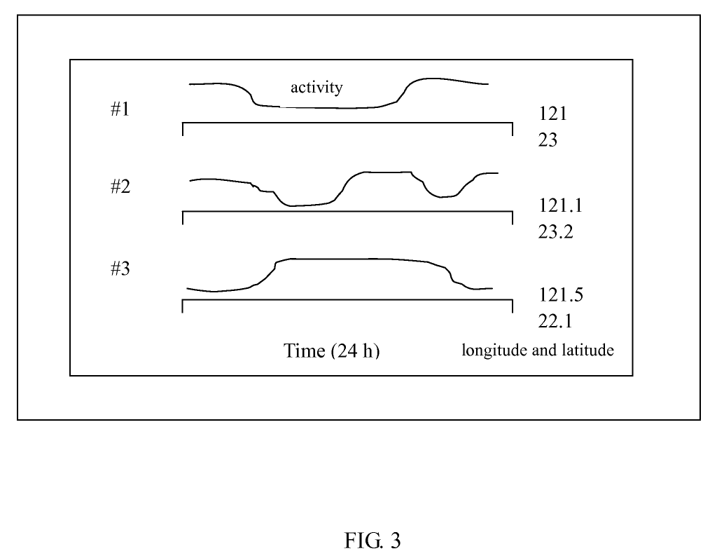

With reference to FIG. 3, it is noted that the information to be analyzed includes the subject's daily exercise cycle and the subjects' location, i.e. latitude and longitude.

With reference to FIGS. 1 and 3, it is noted that the remote host 12 is able to pick up the subject's daily cycle, sleeping cycle, exercise capability within 24 hours and oxygen consumption rate from the gathered subject's continuous information within 24 hours. Based on the gathered information, the remote host 12 is able to tell whether the subject is within normal range and should there be any abnormal situation happened to the evaluated subject, the remote host 12 issues a warning message to appropriate personnel to respond accordingly. Furthermore, the remote host 12 may respectively transmit multiple reminder to each of the activity monitoring devices 111, 112, 113 via the transmission module 121 to remind the evaluated subject to do exercise and/or of daily routines.

As a result, it is notable that the remote host 12 of the preferred embodiment is able to consolidate and analyze the gathered information, as well as track the activity and positioning information of people. When necessary, the remote host 12 is able to transmit messages to the evaluated subject via ear set and remind the evaluated subject to do exercises. Also, the evaluated subject may transmit messages to the remote service personnel as a feedback. Therefore, monitoring to solitary senior citizens and studies on group livings are available.

To sum up, the activity monitoring system and the monitoring device of the preferred embodiment integrate global positioning system, wireless communication module and status sensor modules to provide an automatic message transmission mechanism to the remote host 12 and an inexpensive, durable activity monitoring device.

It should be apparent to those skilled in the art that the above description is only illustrative of specific embodiments and examples of the invention. The invention should therefore cover various modifications and variations made to the herein-described structure and operations of the invention, provided they fall within the scope of the invention as defined in the following appended claims.

What is claimed is:

1. An activity monitoring device adapted to be mounted on a subject to be evaluated and for transmitting information to a remote host, the activity monitoring device comprising:
a global positioning system for providing a positioning information;
a wireless communication module;
a status sensor module including a multi-axle accelerating sensor and a heart rate variability sensor for sensing subject's activity status and bio-status so as to provide at least one activity information related to the subject; and
a microprocessor coupled to the global positioning system, the wireless communication module and the status sensor module,
wherein the microprocessor controls the wireless communication module to send the positioning information and the at least one activity information to the remote host.

2. The device as claimed in claim 1 further comprising a storage unit for storing the positioning information and the at least one activity information such that when the positioning information and the at least one activity information reach a predetermined volume or store in the storage unit in a predetermined time, the microprocessor controls the wireless communication module to transmit the positioning information and the at least one activity information to the remote host.

3. The device as claimed in claim 1, wherein the microprocessor integrates the positioning information and the at least one activity information into an activity monitoring information and the microprocessor controls the wireless communication module to transmit the activity monitoring information to the remote host.

4. The device as claimed in claim 1, wherein the wireless communication module sends a notice signal to the microprocessor when quality of communication at which the subject is located is good and the microprocessor controls the wireless communication module to transmit at least one of the positioning information and the at least one activity information to the remote host.

5. The device as claimed in claim 1 further comprising a display unit for displaying a warning message transmitted by the remote host when abnormal circumstances judged by the remote host happen.

6. The device as claimed in claim 1 further comprising an audio player/receiver for providing a warning message transmitted by the remote host when abnormal circumstances judged by the remote host happen.

7. The device as claimed in claim 1, wherein the activity monitoring device in integrated into a watch, a necklace or an ornamental accessories.

8. The device as claimed in claim 1 further comprising a storage unit for storing predetermined warning information such that when abnormal match between the predetermined warning information and the activity monitoring information happens, the microprocessor controls the wireless communication module to send a warning message to the remote host.

9. The device as claimed in claim 1, wherein the global positioning system and the wireless communication module respectively have a first operation status and a second operation status, the global positioning system and the wireless communication module are in the first operation status normally and the microprocessor judges from the activity monitoring information to determine whether the global positioning system and the wireless communication module enter the second operation status.

10. The device as claimed in claim 9, wherein the first operation status is off and the second operation status is on.

11. An activity monitoring system for monitoring multiple subjects and for transmitting information to a remote host, the activity monitoring system comprising:
a remote host; and
multiple activity monitoring devices to be attached to the subjects and each activity monitoring device including:
a global positioning system, for providing a positioning information;
a wireless communication module;
a status sensor module including a multi-axle accelerating sensor and a heart rate variability sensor for sensing subject's activity status and bio-status so as to provide at least one activity information related to the subject; and
a microprocessor coupled to the global positioning system, the wireless communication module and the status sensor module,
wherein the microprocessor controls the wireless communication module to send the positioning information and the at least one activity information to the remote host.

12. The system as claimed in claim 11, wherein the remote host includes a database for storing the positioning information and the activity information transmitted by the activity monitoring devices, the remote host analyzes the stored positioning information and the activity information.

13. The system as claimed 11, wherein the remote host analyze capability of subjects' activity and exercise scope based on the positioning information and the activity information.

14. The system as claimed in claim 11, wherein the remote host includes a transmission module for periodically transmitting multiple reminders to the activity monitoring devices.
